(19) United States
(12) Patent Application Publication
(10) Pub. No.: US 2004/0260207 A1
(43) Pub. Date: Dec. 23, 2004

(54) Device for measuring distances within body cavities

(76) Inventors: Meir Eini, Nes Ziona (IL); Dov Tamarkin, Maccabim (IL); Judith Sarig, Kfar Saba (IL); Alfons Johannes Baur, Doar Na Megiddo (IL)

Correspondence Address: G.E. Ehrlich, Anthony Castorina, 2001 Jefferson Davis Highway, Suite 207, Arlington, VA, 22202, US

(21) Appl. No.: 10/486,366

(22) PCT Filed:
(86) PCT No.: PCT/IL02/00716

Related U.S. Application Data
(60) Provisional application No. 60/315,720, filed on Aug. 30, 2001.

(51) Int. Cl. A61B 5/103

(52) U.S. Cl. 600/587

(57) ABSTRACT

A distance measuring device (10) operable to measure distances within a body cavity. The device (10) includes a flexible container (12) having a shape that is subject to deformation by pressure exerted on the flexible container; an electronic measuring unit (14) contained in the flexible container to respond quantitatively to deformations in the shape of the container and an output unit (20) for transmitting data from the measuring device to an external data utilization system.

### FIELD AND BACKGROUND OF THE INVENTION

The present invention relates to a device for electrically stimulating muscles and nerves defining and surrounding a bodily cavity and/or for sensing electrical activity of said muscles, and, more particularly, to a device and system utilizing same which are useful in providing biofeedback on muscle behaviour in such a cavity.

Electrical-stimulation (ES) has been found to be effective in increasing muscle strength while biofeedback monitoring of muscular activity is valuable in assessing muscle activity. A particular application thereof is in the treatment of incontinence in which correct pelvic floor muscle control by the patient can be promoted.

U.S. Pat. No. 4,396,019 to Perry, Jr., teaches the use of an electrode-carrying insert which functions in providing the patient with feedback on muscle activity and as such enables the patient to exercise self control over the musculature contributing to urinary incontinence.

U.S. Pat. No. 4,881,526 to Johnson teaches an electrode and controller for preventing female urinary incontinence. The electrode includes an elongated and generally cylindrical carrier having a rounded tip, an extended lip, and a neck of reduced diameter. Motor receptor electrical stimulation signals received from the controller are coupled to the motor electrodes and directly stimulate pelvic floor musculature.

The rigid, non-yielding structure, of the above-described electrode carrying devices presents several disadvantages. Since contact between a wall of the intrabody cavity and an electrode of such devices is of utmost importance for efficient muscle activation, such devices must be fabricated in a variety of sizes to fit a variety of anatomical builds. In addition, the rigid construction of such devices interferes with the physiological movement of an exercising vaginal muscle. Furthermore, the rigidity of the device greatly increases patient discomfort.

To overcome these limitations, U.S. Pat. No. 5,662,699 to Hamedi, teaches of a device which includes a flexible airtight sheath with a resilient skeleton and outer conductive bands which is collapsed by vacuum and inserted into the body cavity. When inflated within the cavity, the skeleton expands the sheath and forces the conductive bands against the body cavity wall thus ensuring optimal contact.

This device is limited to recording muscle activity, no description is provided for its ability to induce muscle stimulation. In addition, although the use of such configuration overcomes the limitations inherent to rigid electrode carrying devices, the need for an inflating/deflating mechanism greatly complicates the fabrication and application of such a device and if air leakage should occur, also its reliability in interpretation of the results.

There is a general tendency of bodily cavities, particularly the vagina and rectum, to expel anything that is inserted therein. A suitable intracavity device must therefore be able to withstand such expulsive tendencies, especially if the device is intended to remain within the cavity for extended periods of time. For biofeedback-based training, extended periods of time are generally necessary. There is thus a widely recognized need for, and it would be highly advantageous to have, an intracavity device capable of electrically stimulating, and/or recording the activity of, the musculature/nervure defining and surrounding the cavity and yet devoid of the above mentioned limitations of prior art designs.

### SUMMARY OF THE INVENTION

According to a first aspect of the present invention there is provided a distance measuring device operable to measure distances within a body cavity, comprising:

(a) a flexible container whose shape is subject to deformation by pressure exerted on said container

(b) an electronic measuring device contained in said flexible container, operable to respond quantitatively to deformations in the shape of said container,

(c) an output unit for transmitting data from said measuring device to an external data utilization system.

The device preferably further comprises an external data-utilization system.

Preferably, said container, when not subject to deforming pressure, is of substantially annular shape.

Preferably, said container, when not subject to deforming pressure, is of a substantially rounded diamond shape.

Preferably, said container comprises a layer of silicon substantially covering and containing said electronic measuring device.

Preferably, said data utilization system comprises a user sensible display.

Preferably, said display comprises a bar display.

Preferably, in said data utilization system comprises a microprocessor.

Preferably, said data utilization system comprises a memory.

Preferably, said data utilization system comprises a data recording facility.

Preferably, said data recording facility comprises a database.

Preferably, said data utilization system comprises a biofeedback system.

Preferably, said data utilization system comprises a calculation module for comparing data recorded at a first time to data recorded at a second time.

Preferably, said data utilization system comprises a trend displayer for displaying to a user trends discerned by said calculation module.

Preferably, said output unit comprises a wire connection operable to transmit data between said measuring device and said external data utilization system.

Preferably, said output unit comprises a radio-frequency data transmission module operable to transmit data between said measuring device and said external data utilization system.

Preferably, said output unit comprises a fiber-optic connection operable to transmit data between said measuring device and said external data utilization system.

Preferably, said output unit comprises an infra-red data transmission module operable to transmit data between said measuring device and said external data utilization system.

Preferably, said electronic measuring device comprises a capacitance measurement tool operable to respond quantitatively to changes in capacitance of a capacitance module, said change in capacitance being induced by variations in the shape of said container.

Preferably, said electronic measuring device comprises an inductance module comprising a tuned oscillator including a coil, and further comprising a metallic element flexibly connected to said coil, said flexible connection being such that changes in shape of said container change a spatial relationship between said metallic element and said coil, thereby modifying inductance of said coil, thereby modifying frequency and phase of oscillation of said tuned circuit.

Preferably, said inductance module further comprises a quantification module operable to respond quantitatively to said modification of frequency.

Preferably, said inductance module further comprises a quantification module operable to respond quantitatively to said modification of phase.

Preferably, said electronic measuring device comprises a signal strength measurement sensor comprising

(a) a signal generator operable to generate a generated signal receivable by a signal receiver, and

(b) at least one signal receiver,

said at least one signal receiver being flexibly connected to said signal generator in such a manner that changes to the shape of said container necessarily change a spatial relationship between said signal generator and said signal receiver, thereby modifying a strength of said signal as received by said signal receiver.

Preferably, said signal generator is a generator of electronic signals.

Preferably, said signal generator is operable to generate a signal of a type selected from a group including pulse signals, wave signals, modulated pulse signals, modulated wave signals, changing pulse signals, changing wave signals, and changing frequency signals.

Preferably, said signal generator is an infrared transmitter and said signal receiver is an infrared receiver.

Preferably, said signal generator is a light transmitter and said signal receiver is a light receiver.

Preferably, said signal generator is a permanent magnet and said signal receiver is a magnetic sensor.

Preferably, said magnetic sensor is a Hall effect semiconducter receiver.

Preferably, said signal generator is an electro-static field generator utilizing a plate antenna, and said signal receiver is an electro-static field sensor utilizing a plate antenna.

Preferably, said signal generator is an ultrasound sound generator, and said signal receiver is an ultrasound sound sensor.

Preferably, said signal generator is a loudspeaker, and said signal receiver is a microphone.

Preferably, said signal generator comprises a very low radiation isotope, and said signal receiver is a radiation sensor operable to measure an amount of received radiation.

Preferably, said electronic measuring device comprises a compressible gas-filled structure containing a variable-resistance device whose electrical resistance varies as a function of pressure of gas in said compressible gas-filled structure.

Preferably, said electronic measuring device comprises a variable resistor whose resistance is a function of mechanical exerted on said resistor.

According to a second aspect of the present invention there is provided a muscle strength measuring device for measuring forces exerted on said device by muscles of a body while said device is inserted in a cavity of said body, the device comprising:

(a) a flexible container sized and shaped to fit into a body cavity, said container being operable to undergo shape deformation in response to pressure exerted on said body by contraction of muscles constituting or adjacent to said body cavity, and

(b) an electronic mechanism at least partially contained in said container, operable to measure said deformation of said container in response to said pressure.

According to a third aspect of the present invention there is provided a distance measuring device for measuring distances within a body cavity, the device comprising:

(a) a flexible container sized and shaped to fit into a body cavity, said container being operable to undergo shape deformation in response to pressure exerted on said body by walls of said body cavity, and

(b) an electronic mechanism at least partially contained in said container, operable to measure said deformation of said container in response to said pressure.

The device preferably further comprises electrodes for electrically stimulating muscles of said body.

According to a fourth aspect of the present invention there is provided a cavity measuring device operable to measure changes within a body cavity, comprising:

(a) a flexible container whose shape is subject to deformation by pressure exerted on said container

(b) an electronic measuring unit contained in association with said flexible container, operable to respond quantitatively to changes within said cavity,

(c) an output unit for transmitting data from said measuring device to an external data utilization system.

Preferably, said change is a pressure change.

Preferably, said cavity is an animal body cavity and said changes comprises changes in pressure exerted by musculature surrounding said cavity.

Preferably, said electronic measuring unit comprises at least one sensor located outwardly of said device.

Preferably, said electronic measuring unit comprises at least one sensor located in a cavity within said device.

Preferably, said cavity is generally expulsive of foreign objects introduced therein, said flexible container being adapted to deform within said cavity upon being pressurized by said musculature such as to retain itself within said cavity.

According to a fifth aspect of the present invention there is provided a method for biofeedback training of a user, comprising

(a) inserting into a body cavity a muscle strength measuring device operable to measure forces exerted on said device by muscles of a body while said device is inserted in said body cavity, the device comprising:

(i) a flexible container sized and shaped to fit into a body cavity, said container being operable to undergo shape deformation in response to pressure exerted on said body by contraction of muscles constituting or adjacent to said body cavity,

(ii) a sensor mechanism at least partially contained in said container, operable to measure said deformation of said container in response to said pressure, and

(iii) a biofeedback mechanism operatively associated with said sensor mechanism to provide user sensible feedback of said deformation, and

(b) using said biofeedback to guide muscle exercising.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a perspective view of a device for stimulating muscles and nerves defining or surrounding the cavity and/or sensing activity of said muscles, according to the teachings of the present invention;

FIG. 2 a perspective view of a power and control unit attachable to the device described in FIG. 1;

FIG. 3 is a side view of the device shown in FIG. 1 connected to the power and control unit shown in FIG. 2;

FIGS. 4a-b demonstrate the process of inserting and positioning the device for stimulating muscles and nerves defining or surrounding the intravaginal cavity, and/or sensing activity of said muscles, according to the teachings of the present invention;

FIG. 5 shows a system in accordance with the teachings of the present invention; and

FIGS. 6-15 show varying levels of detail of embodiments of an device according to the present invention,

### DESCRIPTION OF THE PREFERRED EMBODIMENTS

The present embodiments comprise an insertion device, for example an intravaginal or intrarectal device, which can be used to electrically stimulate the muscles and nerves defining and surrounding the cavity and/or sense, record and report the muscles electrical or mechanical activity. Specifically, the present embodiments can be used to provide information to a biofeedback system.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

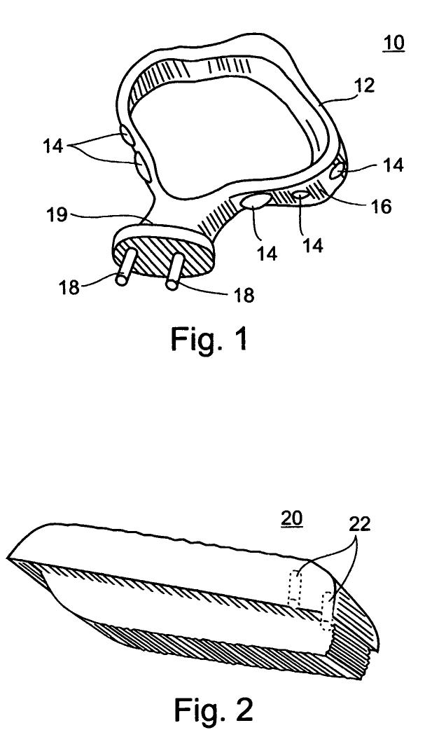

Referring now to the drawings, FIG. 1 illustrates one possible configuration of a device for stimulating, and/or sensing electrical activity of, muscles and nerves defining and surrounding a bodily cavity of an individual, which is referred to hereinunder as device 10.

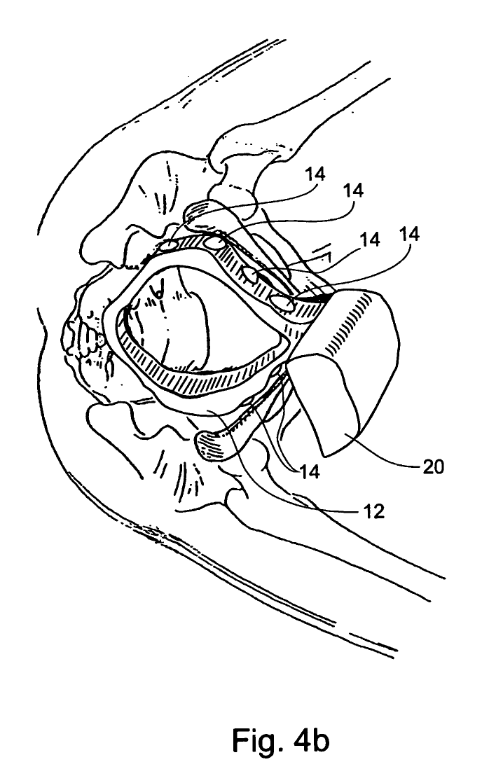

Device 10 includes a body 12. Body 12 is constructed such that when contracted and positioned within an cavity of the individual (see FIGS. 4a-b), body 12 self expands to conform to a contour of the cavity. This memory (self expansion) property of body 12 can be achieved by fabricating at least a portion of body 12 from an elastic material having such memory, such as, but not limited to, silicon, rubber, latex, or alternatively, by providing various sprigged hinge points along body 12 which allow contracting of body 12 against a force of a spring, thus allowing body 12 to self expand following contraction.

In one preferred embodiment, a hollow silicon mold is used. Electronics of the kind described in detail hereinbelow is inserted therein and then the whole is sealed, typically by fusion with cold silicon.

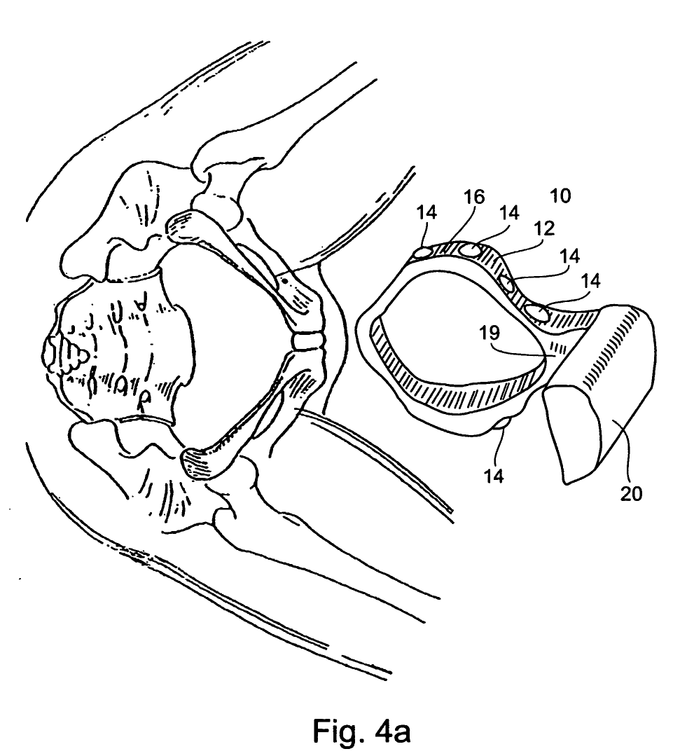

As best seen in FIG. 3, device 10 further includes at least one pair of electrodes 14. In one embodiment of the present invention some or all of electrodes 14 serve for providing an electrical current to the walls of the cavity of the individual. In another embodiment of the present invention some or all of electrodes 14 serve for recording electrical activity of the muscles defining the walls of the cavity of the individual. In either case, electrodes 14 are preferably attached to an exterior surface 16 of body 12. Thus, when body 12 is positioned within the cavity, each electrode 14 is biased against a wall of the cavity to thereby maintain electrical contact with the wall.

Preferably, device 10 includes several pairs of electrodes 14, each pair being attached to a specific region of exterior surface 16. According to this preferred configuration of device 10, each pair of electrodes is biased against (contacts) a specific region of the cavity wall when device 10 is positioned within the cavity.

As is further detailed in the Examples section which follows, this configuration of device 10 of the present invention is particularly advantageous since it enables specific stimulation (electrical) of specific regions of the cavity wall according to a predetermined stimulation pattern.

To maintain optimal electrical conductance, the portion of electrodes 14 which maintains contact with the cavity wall is fabricated from a material such as, but not limited to, gold and platinum, or any other highly conductive material including metal alloys and composites.

It will be appreciated that when expanding to its relaxed state, body 12 causes reduction (anatomical repositioning) of prolapsed tissue and therefore, to the benefit of more physiological muscular exercising. The flexibility of body 12 in itself, as opposed to rigid constructions, also adds to achieving more physiological muscular exercising.

The electrical current provided from electrodes 14 serves for electrically stimulating muscles and/or nerves defining and surrounding the cavity.

Since the position of these muscles, in relation to the cavity varies from one individual to the next, placement of electrodes 14 upon exterior surface 16 is of crucial importance when wanting to achieve maximal stimulation/activity records in each individual. It will further be appreciated that although optimal placement of electrodes 14 can be achieved by testing the device on a large number of individuals, such anatomical limitations can be traversed by placing several pairs of electrodes 14 upon exterior surface 16 as is mentioned hereinabove thus enabling the activation of one or more specific pairs of electrodes 14 according to individual anatomical built. This may also be advantageous in cases wherein muscles of one side of the cavity are stronger then the muscles of the other side as is typically the case in birth related incontinence. Thus, muscle stimulation with the device of the present invention may be applied preferentially or solely to the weak muscles, as opposed to all of the muscles surrounding and defining the vaginal cavity.

As shown in FIGS. 1 and 3, one possible configuration of body 12 is a substantially rounded diamond shaped frame of approximately 5-7 cm in length and 7-9 cm in width in its relaxed state. In this configuration, electrodes 14 are preferably positioned on exterior surface 16 of the members defining such a frame. The construction of body 12 is selected such that when relaxed it is 1.3-1.7, preferable 1.5, times shorter than when fully contracted. Similarly, the width of body 12 is selected such that when relaxed is at least 1.3-1.7, preferable 1.5, times larger than when fully contracted. This enables body 12 to be utilizable in a wide range of anatomical builds.

Device 10 further includes connectors 18, which serve for electrically interfacing with a power and control unit, which is further described below.

Preferably, connectors 18 are positioned on a neck 19 of body 12 such that when body 12 is positioned within the cavity and attached to the power and control unit, the power and control unit is positioned outside of the cavity.

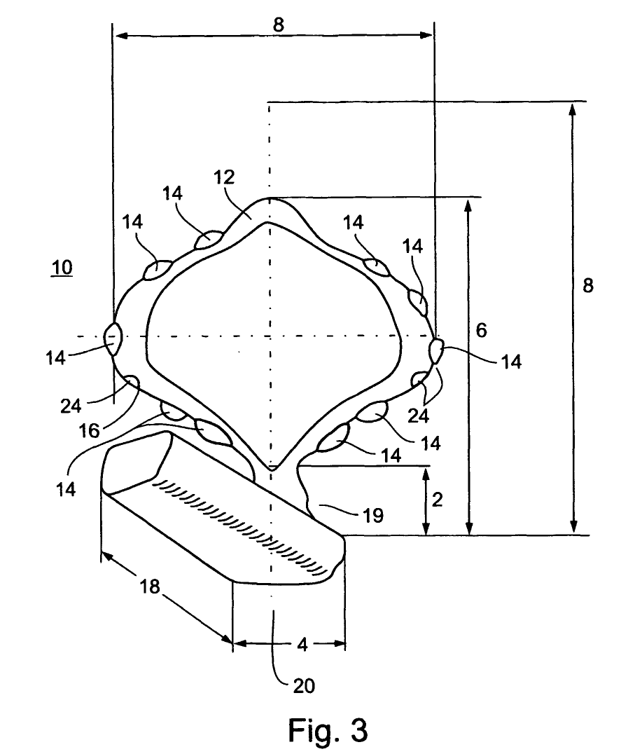

Thus, as is specifically shown in FIG. 2, device 10 further includes a power and control unit 20, which serves for providing electrical currents or to electrodes 14 and/or for creating a potential difference therebetween. Power and control unit 20 includes receptacles 22, which serve for interfacing with connectors 18 described above.

Power and control unit 20 preferably also includes an attachment mechanism for securing power and control unit 20 to the underwear or body of the individual when device 10 is in use. This ensures that body 12 does not translate or rotate inside the cavity when in use, thus guaranteeing optimal electrical contact and stimulation of the desired muscles. Such attachment mechanism can include Velcro™ fasteners for securing power and control unit 20 to the underwear or pubic hair of the individual, or it can include sticky tape or suction cups for securing power and control unit 20 against the body of the individual.

Power and control unit includes a power source, such as a battery, a control unit for controlling the output from the power source and the required circuitry. Power and control unit 20 preferably also includes a processing unit as is further detailed hereinbelow.

Power and control unit 20 preferably also includes exterior controls for controlling the intensity, frequency and duration of the electrical current provided to electrodes 14. In device 10 which includes more than one pair of electrodes 14, such exterior controls can also be used to separately control each pair of electrodes 14, such that an intensity, frequency and/or duration of an electrical current delivered from each pair of electrodes 14 can be independently controlled. In addition, power and control unit 20 can also be preprogrammed to deliver a preset pattern of stimulation via one or more pairs of electrodes 14.

For example, an individual or a treating physician can set the power and control unit according to individual needs, or activate a preset program of stimulation in order to effectively stimulate and thus contract and exercise the musculature defining or surrounding the cavity. The Examples section, which follows, details various stimulation regimens which are suitable for incontinence treatment and which can be provided by device 10 of the present invention

Preferably, electrical current is provided from power and control unit 20 in an intermittent pattern, which includes 1-20, preferably 2-10 seconds intervals each providing alternating current spaced by 2-40, preferably 4-20 seconds of rest intervals. Generally a pulse width of 100-200 microseconds is used with intervals to give a frequency of 5-90 Hz.

As shown in FIG. 3, according to a preferred embodiment of the present invention device 10 further includes at least one sensor 24 (two are shown in FIG. 3), which is preferably attached to exterior surface 16 of body 10. Sensor(s) 24 serve for sensing muscle activity of the muscles defining and surrounding the cavity. Such sensing can be either: prior to, during, or following stimulation of these muscles via electrodes 14.

Sensor(s) can be either pressure sensors, or sensors which are capable of sensing muscle electrical activity (e.g., surface electromyography sensors). It will be appreciated that, in the latter case a grounding electrode must be included in device 10.

It will likewise be appreciated that pressure sensors do not need to be mounted on the exterior of the device but may likewise be mounted internally of the device.

It will likewise be appreciated that any given device may have either or both of stimulatory electrodes and sensors, depending on the application.

It is further pointed out that, according to the electronic circuitry to be described in detail below, a sensory signal indicative of separate muscular activity at separate locations on the device is obtainable.

In any case, sensors 24 serve to evaluate muscle activity prior to, during or following treatment to thereby serve as either a basis for a treatment regimen or as feedback to a treatment regimen.

According to one preferred embodiment of the present invention, sensors 24 interface with power and control unit 20 via connectors 18 or any other dedicated connectors preferably positioned at neck 19. To this end, sensors 24 are provided with power from power and control unit 20 and also communicate information relating to muscle activity to power and control unit 20.

Such information received by power and control unit 20 can be processed by a processing unit contained therein and the processed information utilized to automatically adjust or set the intensity and/or duration of the electrical current provided from power and control unit 20 to electrodes 14.

It will be appreciated that power and control unit 20 can also include a memory device and ports for interfacing with a personal computer. Thus, sensor information of each treatment session, which is collected by power and control unit 20, can be uploaded onto a computer for storage and/or further analysis.

The above-described configuration of device 10 of the present invention is particularly advantageous since it enables device 10 to set the most suitable course of treatment for an individual.

Alternatively, the information provided by the sensor can be transmitted to an extra-corporeal unit for analysis via a transmitter included within sensor(s) 24 or body 12 or preferably within power and control unit 20.

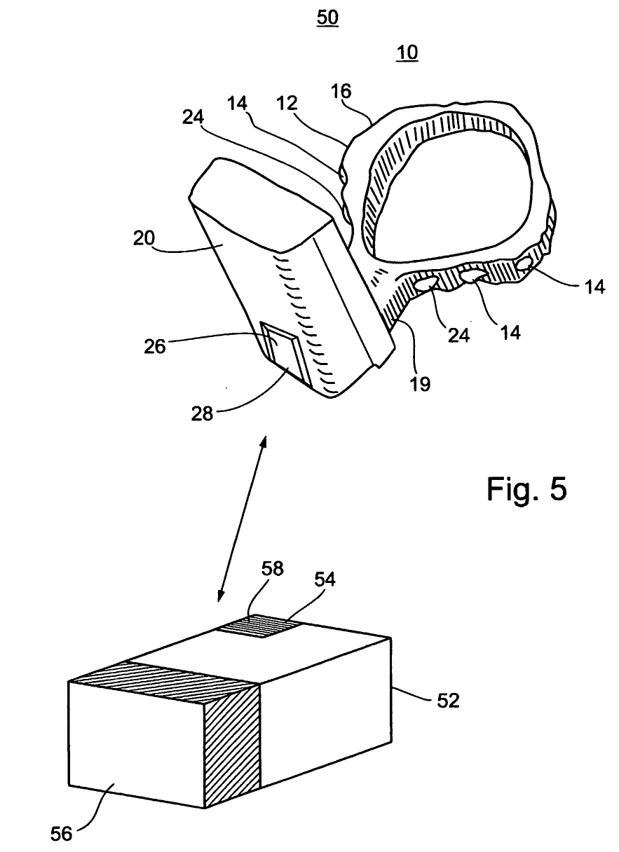

Thus, according to another aspect of the present invention and as shown in FIG. 5, device 10 of the present invention can form a part of a system for stimulating muscles and nerves defining and surrounding an cavity of an individual, which is referred to hereinunder as system 50.

System 50 includes device 10 as described above, which according to this aspect of the present invention includes a transmitter 26 preferably positioned on or in power and control unit 20. Transmitter 26 serves for transmitting a signal receivable outside the body, which signal includes data pertaining to muscle activity sensed by sensor(s) 24.

System 50 also includes an extracorporeal unit 52 which serves for processing the signal received from transmitter 26 to thereby determine duration or intensity of the electrical current provided to electrodes 24 from power and control unit 20. To this end, extracorporeal unit 52 includes a receiver 54 and a processing unit 56 (e.g., a personal computer).

Preferably, power and control unit 20 of device 10 also includes a receiver 28. Thus, following processing of the signal, received from transmitter 26, extracorporeal unit 52 preferably transmits, via a transmitter 58, a command signal receivable by receiver 28, which command signal determines the duration or intensity of the electrical current provided to electrodes 24 from power and control unit 20.

It will be appreciated that the above-described configuration of system 50 is advantageous since extracorporeal unit 52 enables a more accurate processing of the information collected by sensor(s) 24 to thereby more accurately determine a suitable course of treatment. In addition, this configuration of system 50 also enables processing and storing of information collected from several treatment sessions conducted over any period of time of say weeks or months. Furthermore, statistical analysis of information collected from several individuals can be utilized to improve treatment regimens or to improve the construction of device 10.

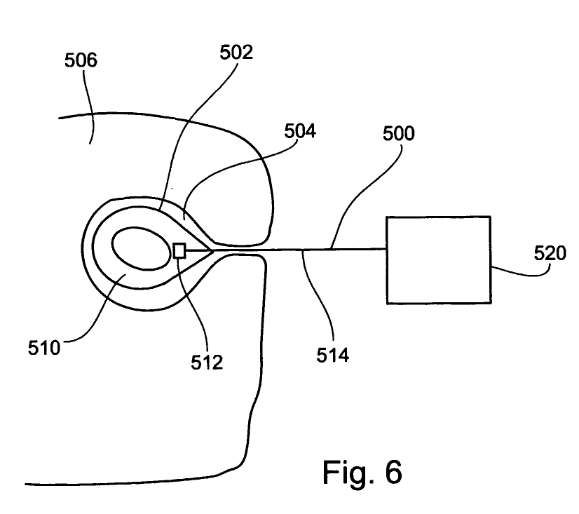

Reference is now made to FIG. 6, which is a therapeutic system 500 according to an embodiment of the present invention. System 500 is shown inserted in a cavity 504 of a body 506, and may be used for measuring for measuring forces acting to compress body cavity 504, such as forces generated by the contraction of muscles constituting or in proximity to cavity 504.

System 500 comprises a measurement device 502, preferably of annular shape. Alternatively, the shape of device 502 is a modified annular form characterized by a rounded diamond shape. Device 502 is flexible, and its shape is capable of distortion in response to pressures exerted thereupon.

Device 502 comprises a flexible container 510, preferably of silicon, and an electronic measuring device 512 at least partially contained therein. Measuring device 512 is operable to respond quantitatively to deformations in the shape of container 510.

Device 502 further comprises an output unit 514 for transmitting data from measuring device 512 to an data utilization system 520. Data utilization system 520 is preferably external to device 502.

Output unit 514 is any apparatus operable to transmit data between measuring device 512 and data utilization system 520. In a preferred embodiment, output unit 514 is simply a wired connection between measuring device 512 and data utilization system 520. In alternative constructions, output unit 514 is a data transmission device such as a radio-frequency data transmission module, a fiber-optic connection, or an infra-red data transmission module.

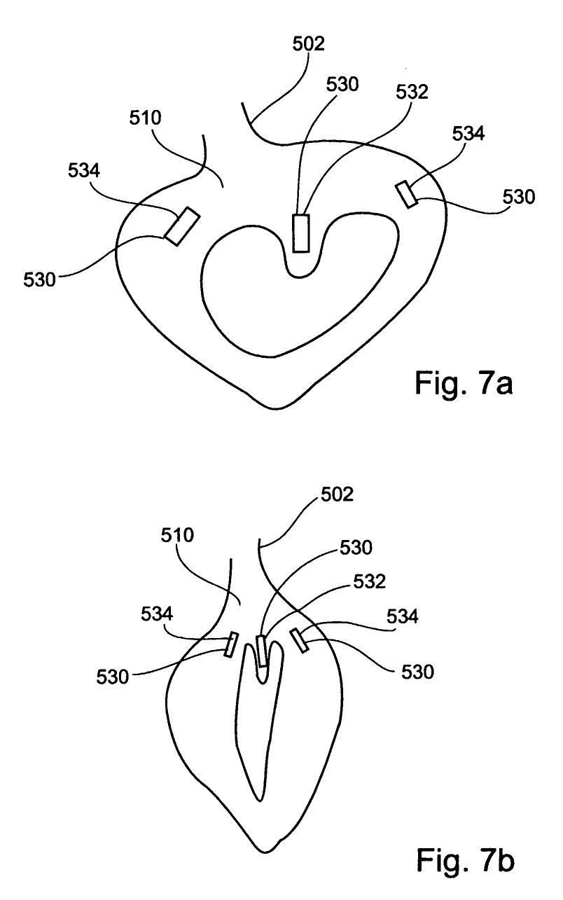

Reference is now made to FIGS. 7a and 7b, which comprise a first alternative construction of device 502, according to a preferred embodiment of the present invention. In the embodiment of FIG. 7a, electronic measuring device 512 comprises a signal strength measurement sensor 530. Signal strength sensor 530 preferably comprises a signal generator 532 operable to generate a signal, and at least one signal receiver 534, operable to receive the signal generated by signal generator 532.

Signal receiver 534 is flexibly connected to signal generator 532. In a preferred embodiment, flexible connection is provided by flexible container 510, which is constructed of a semi-rigid yet flexible material. The shape of container 510 is subject to change and distortion in response to pressures exerted on container 510 from external sources, yet the material of container 510 will tend to return to its shape when such external pressures are relaxed.

In consequence of the flexible connection between signal generator 532 and signal receiver 534, changes in external forces acting on container 510, and consequent changes in the shape of container 510 as a result of those forces, causes a change in the spatial relationship between signal generator 532 and signal receiver 534. The signals generated by signal generator 532 are of a strength such that changes to the distance, or other aspects of the spatial relationship, between generator 532 and receiver 534 affects the strength of the signal as received by receiver 534. Thus, for example, in a particular configuration, external forces compressing portions of container 512 might cause generator 532 to move closer to receiver 534, thereby increasing the strength of the received signal detected by receiver 534. Similarly, relaxation of external forces, allowing container 510 to return to its relaxed form, might then move generator 532 further from receiver 534, thereby weakening the signal received by receiver 534.

In FIG. 7a, device 502 is shown with container 510 in its relaxed state, with signal receivers 534 relatively distant from signal source 532. FIG. 21b, by way of contrast, shows device 502 as it is when container 510 is compressed by external forces, causing signal receivers 534 to be moved closer to signal source 532.

In a preferred embodiment, generator 532 is a generator of electronic signals. Typically, signals generated by generator 532 may be pulse signals, or wave signals, or modulated pulse signals, or modulated wave signals, or changing pulse signals, or changing wave signals, and changing frequency signals.

In alternative preferred constructions, generator 532 is an infrared transmitter and signal receiver 534 is an infrared receiver.

In an additional alternative preferred construction, generator 532 is a light source and receiver 534 is a light-sensitive sensor.

In an additional alternative preferred construction, generator 532 is a permanent magnet generating a magnetic field, and signal receiver 534 is a magnetic sensor, such as a Hall effect semiconductor receiver.

In an additional alternative preferred construction, generator 532 is an electro-static field generator utilizing a plate antenna, and signal receiver 534 is an electro-static field sensor utilizing a plate antenna.

In an additional alternative preferred construction, generator 532 is an ultrasound sound generator, and receiver 534 is an ultrasound sound sensor. In a further preferred embodiment, generator 532 is a sound source such as a loudspeaker, and receiver 534 is a sound sensor such as a microphone.

In an additional alternative preferred construction, generator 532 is a very low radiation isotope, and signal receiver 534 is a radiation sensor operable to measure an amount of received radiation.

Attention is now drawn to FIG. 7c, which is a block diagram presenting a simplified schematic of an embodiment according to the present invention, wherein is shown electrical connections consistent with the embodiment presented by FIGS. 7a and 7b, and showing an additional amplification stage provided by signal amplifiers 535 for amplifying signals received by signal receiver 534, and further presenting external data utilization system 520, here implemented as a bar graph display 524.

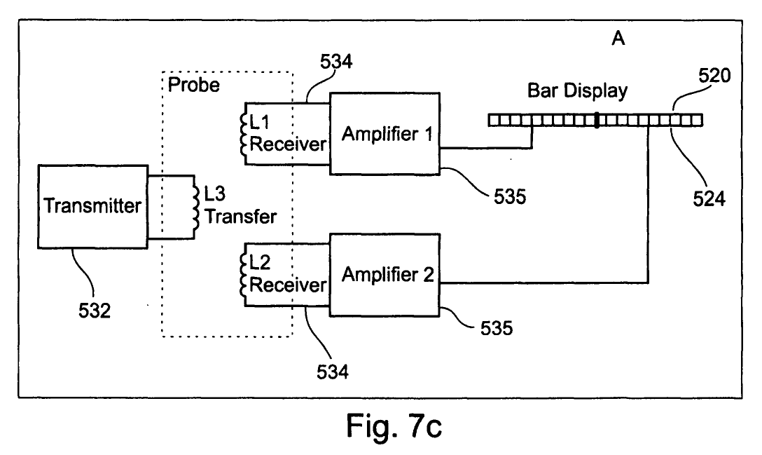

Attention is now drawn to FIGS. 8a and 8b, which present an additional alternative construction of a device according to a preferred embodiment of the present invention. A variable resistance element 540 presents an electrical resistance which varies as a function of stress or pressure applied to element 540. In the construction presented in FIG. 22a, forces tending to compress container 510 will tend to bend variable resistance element 540 which is a stress sensitive resistor 544, thereby changing its resistance. A standard voltage is applied to variable resistance element 540, and voltage drop across variable resistance 540 is measured by a voltmeter, or current in the circuit measured by an ammeter, thereby obtaining a measure of the compression forces to which container 510 is being subjected or of the size to which container 510 has been compressed.

In an alternative construction presented in FIG. 8b, container 510 entirely contains a gas-filled ballon-like partition 546, which also contains variable resistor 540 which is a pressure-sensitive resistor 548. Compression of container 510 by outside forces causes compression of the gas contained in partition 546. This change in pressure causes a change in electrical resistance of pressure-sensitive resistor 548. A standard voltage is applied to resistor 548, and voltage drop across resistor 548 is measured by a voltmeter, or current in the circuit is measured by an ammeter, thereby obtaining a measure of the compression forces to which container 510 is being subjected, and thereby, indirectly, a measure of the size to which container 510 has been compressed.

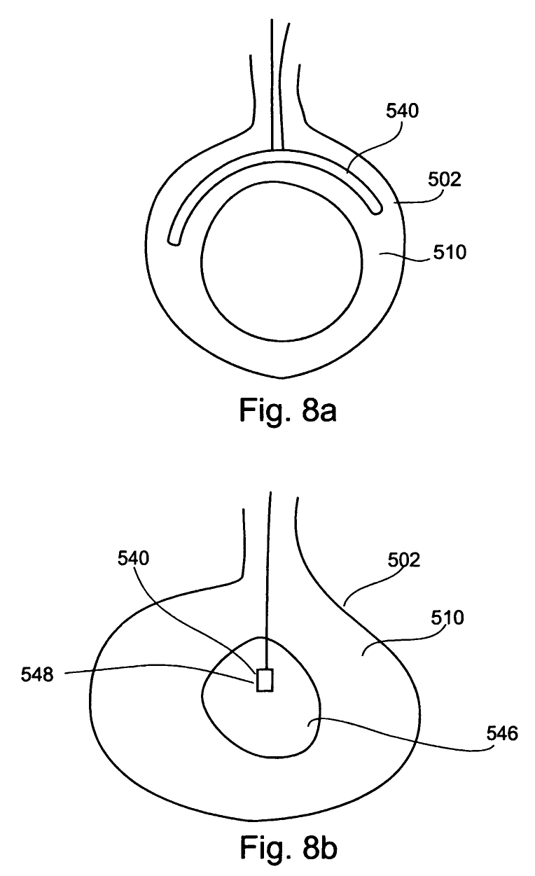

Attention is now drawn to FIGS. 9a and 9b, which present alternative constructions for a measuring device according to the present invention, wherein forces exerted on the device 502 are measured by changes in capacitance of a pressure-sensitive capacitance device.

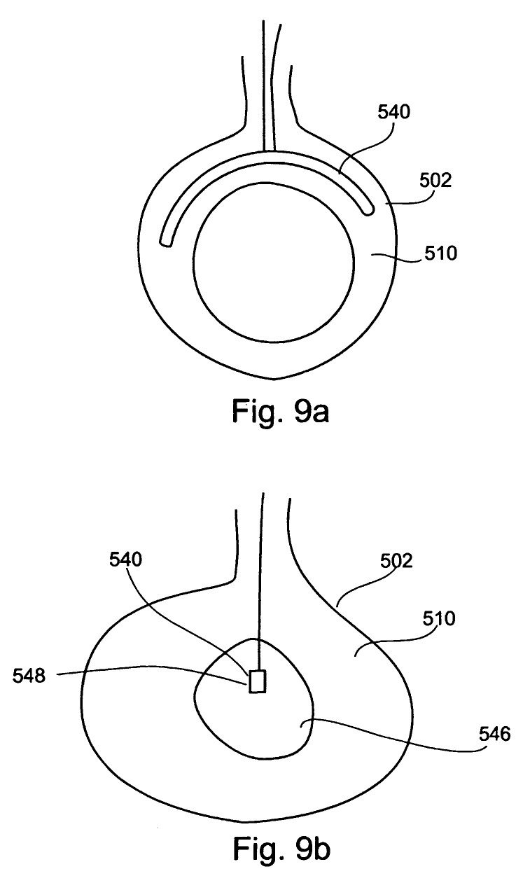

FIGS. 10a and 10b present measuring device 502 wherein electronic measuring module 512 comprises a capacitance measurement tool 550 operable to respond quantitatively to changes in capacitance of a capacitance module 552. Capacitance module 552 is designed and constructed to have a capacitance that varies as a function of pressure applied to module 552. Capacitance measurement tool 510 is situated within container 510 an a manner which ensures that pressure applied to container 510, and causing deformation of the shape of container 510, applies pressure to capacitance module 552.

In the embodiment presented by FIG. 10a, capacitance measurement tool 550 is embedded in container 510 in such a manner that distortions of the shape of container 510 have the effect of creating a lateral squeeze of capacitance module 552. The effect of being squeezed is to modify the capacitance of module 552, which change of capacitance is read and reported by capacitance measurement tool 550.

An alternative construction pictured in FIG. 10b is similar to the construction presented in FIG. 8b, in that a gas-filled partition 546 contains a capacitance module 552 implemented as pressure-sensitive capacitance module 553. Pressure on container 510 has the effect of compressing partition 546, raising the pressure of a gas contained therein, which change of pressure results in a change of capacitance of module 552, which is then read and reported by tool 550.

FIGS. 10c and 10d present, by way of example, alternative constructions for capacitance module 552. In the embodiment of FIG. 10c, a first set of plates 554 is interleaved with a second set of plates 556. Plates 554 and 556 are able to move with respect to each other. Under lateral pressure such as is represented by arrows 558, plates 554 and plates 556 both slide towards the center of the device, thereby increasing an area of interleaving of plates 554 and 556, thereby increasing the capacity of module 552, which change of capacitance is read and reported by tool 550.

In the alternative construction presented in FIG. 10d, a first set of plates 554 and a second set of plates 556 are separated by a spongy material 559. Spongy material 559 is compressible under pressure. Pressure applied to module 552 causes compression of material 559, thus bringing plates 554 into greater proximity with plates 556, thereby increasing capacitance of module 552, which change of capacitance is read and reported by tool 550.

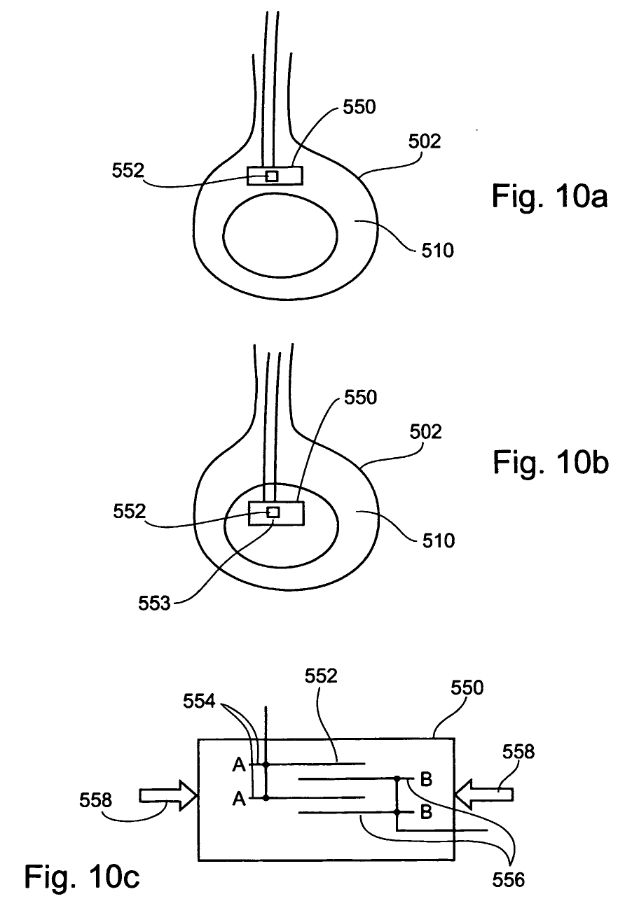

Attention is now drawn to FIG. 11, which is a simplified schematic of an additional preferred embodiment according to the present invention. In FIG. 24, electronic measuring device 512 comprises an inductance module 560. Inductance module 560 comprises a tuned oscillator 562 including a coil 564, and further comprises a metallic element 566 electrically isolated from, but flexibly connected to, coil 254.

The flexible connection between metallic element 566 and coil 254 is such that changes in the shape of container 510 tend to cause modification of the spatial relationship between metallic element 566 and coil 254.

Modification of the spatial relationship between metallic element 566 and coil 254, and in particular modifications which cause metallic element 566 to approach nearer to coil 254 or alternatively to recede further from coil 254, cause, according to well-known laws of physics, changes in the inductance of coil 254.

Since coil 254 is part of tuned oscillator 562, modifying inductance of coil 254 modifies the tuning of tuned oscillator 562. Consequently, changes or deformations in the shape of container 510 under the influence of external forces such as pressure exerted on container 510 by muscle groups near a body cavity in which device 502 is inserted, produce changes in phase and frequency of oscillations of tuned oscillator 252.

Inductance module 560 further comprises a quantification module 568 operable to respond quantitatively to such a modifications of phase, or of frequency, of oscillations of tuned oscillator 252.

Quantification module 568 preferably comprises a reference oscillator 570 for providing a stable reference oscillation, which is used by a comparator 572 operable to compare an oscillation of reference oscillator 570 with an oscillation of tuned oscillator 562. Using comparator 572, quantification module 568 is operable to quantify differences in phase between oscillations of tuned oscillator 562 and oscillations of reference oscillator 570. Alternatively, using comparator 572, quantification module 568 is operable to quantify differences in frequency between oscillations of tuned oscillator 562 and oscillations of reference oscillator 570. Further alternatively, quantification module 568 may be operable to quantify both differences of phase and differences of frequency between oscillations of oscillators 562 and 570.

Quantification module 568 is presented in FIG. 11 as being physically external to container 510, yet in alternate construction, some or all elements of quantification module 568 may be contained within container 510.

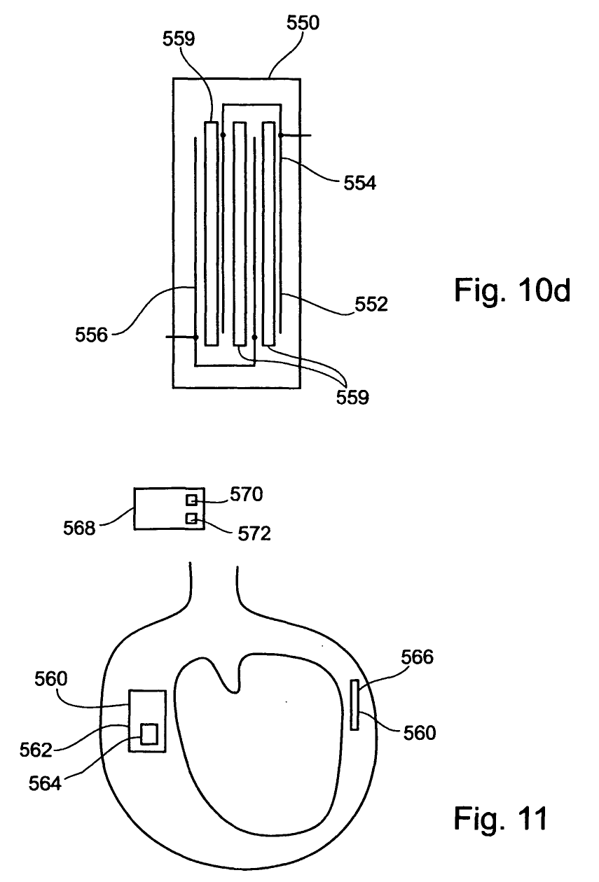

Reference is now made to FIG. 12 which is a simplified circuit diagram for use with the pressure sensing embodiment of the present invention. In FIG. 12 two oscillators 562 have variable frequencies depending on measured pressure of the device. The measured pressure may for example movement of iron element 566 relative to coils 564. A further, stable, oscillator 570 provides a reference signal. Comparators 568 are connected between each of the variable oscillators 562 respectively and the stable oscillator 570 to provide a comparative output indicative of variation in the oscillation rate. At its most simple, comparator 568 may be nothing more than an EXOR gate, the voltage or current of the EXOR output giving an indication of the pressure. The outputs of the comparators 568 are preferably connected to a bar display 580. The use of a bar display is particularly preferred because it provides results which are immediately apparent to the untrained eye. The bar display thus provides a form of very simple biofeedback in that the user is able to determine from the lights on the bar display whether she is succeeding in a given exercise.

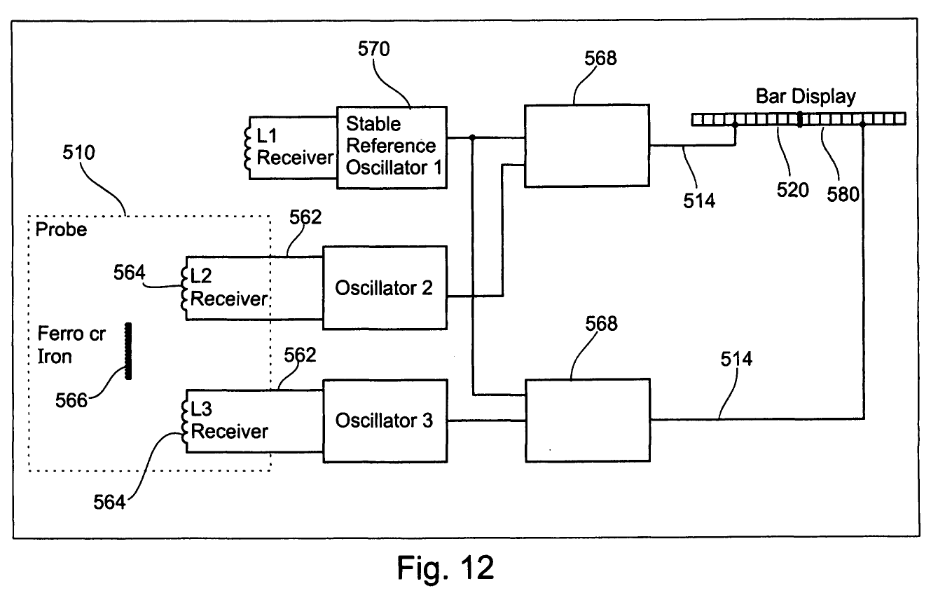

Attention is now drawn to FIG. 13, which is a circuit diagram at component level showing in exemplary form how part of the diagram of FIG. 12 may be realized. FIG. 13 is simplified in that only a single variable oscillator is shown.

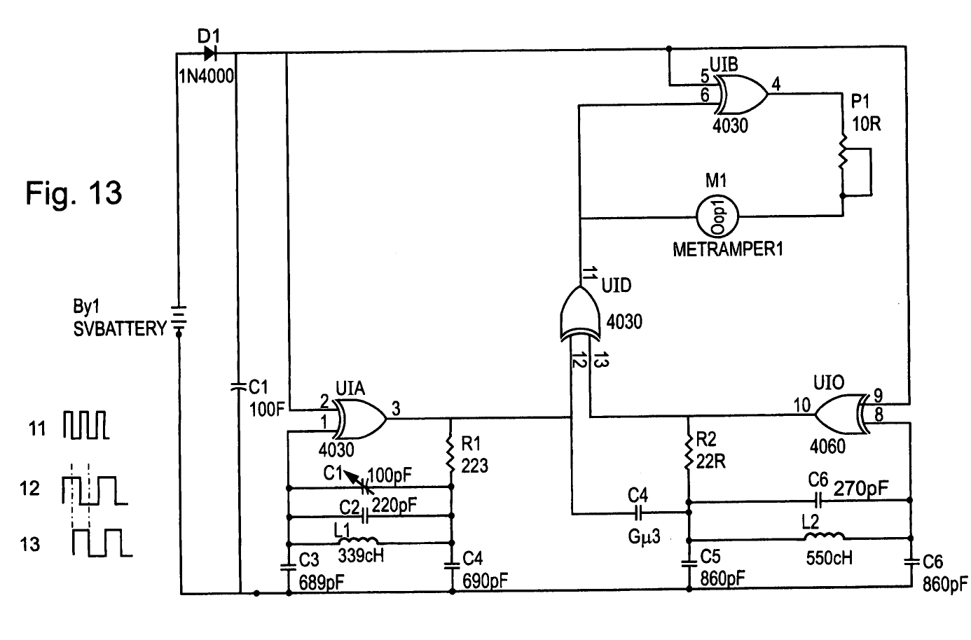

Attention is now drawn to FIG. 14, which is a simplified block diagram presenting components of a data utilization system operable to utilize data provided by a distance measuring device, according to preferred embodiments of the present invention.

FIG. 14 presents a data utilization system 520 operable to receive and utilize data generated by a measuring device 502.

In a preferred embodiment, data utilization system 520 comprises a display 590, typically comprising a visual display 592 and optionally additionally comprising an auditory display 594. Visual display 592 is typically useful to a user such as a medical practitioner utilizing device 502 for such purposes as medical diagnosis of a patient, or for monitoring progress of a medical treatment. Both visual display 592 and auditory display 594 are typically useful for providing feedback to a trainee monitoring his own body processes during a biofeedback training session. In a preferred embodiment, visual display 592 is a bar graph display 524.

In a preferred embodiment, data utilization system 520 further comprises a data recording facility 606 comprising mechanisms for storing data and for storing results of calculations. In a preferred embodiment, data utilization system 520 further comprises a calculation facility 608 for making calculations based on stored data or on data received from device 502.

Calculation facility 608 optionally comprises a microprocessor 596 operable for making calculations.

Data recording facility 606 comprises a memory 598 operable to store data. Stored data is typically utilized as a basis for calculations made by calculation facility 608, and may also be further used for delayed display.

Memory 598 preferably comprises a ram memory 600 for short-term storage and a hard disk 602 for long-term storage, and may optionally further comprise a flash memory 610, a bubble memory 612, a CD reader and recording device 614, a DVD reader and recording device 616, or any similar data storage device able to function as a data recording facility.

In a preferred embodiment, data recording facility 606 comprises a database 620.

In a preferred embodiment, data stored in database 620 during a first treatment of a patient may be compared by calculation facility 608 to data received from device 502 during subsequent treatment of a patient, the results of this comparison being displayed by display 590 in the form of a graphic comparison or in a form which emphasizes trends of development in the received data over time.

In a further preferred embodiment, data utilization system 520 comprises a biofeedback training system 622, operable to provide feedback to an operator regarding measurements of measured by device 502 consequent on voluntary actions by the user, thereby providing a reinforcement system for facilitating learning of voluntary control of selected body processes by the user.

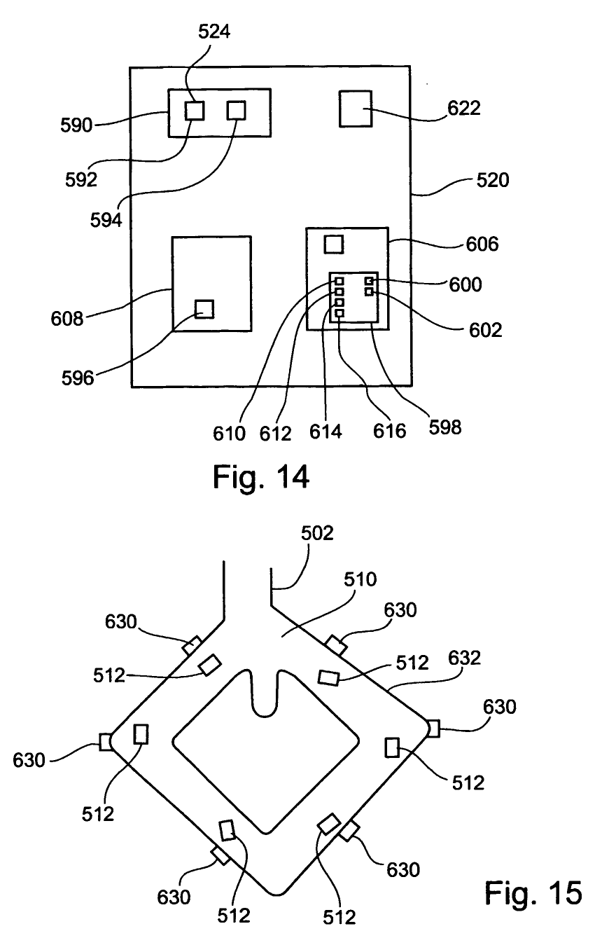

Attention is now drawn to FIG. 15, which is a simplified schematic showing additional preferred embodiments of a distance measuring system according to the present invention.

FIG. 15 presents a distance measuring device 502, presented here as having a modified annular form characterized by a rounded diamond shape. In a preferred embodiment presented in FIG. 15, device 502 comprises a plurality of electronic measurement devices 512. Distribution of a plurality of measurement devices 512 within device 502 presents the advantage that device 502, so configured, is enabled to simultaneously measure distances in a plurality of directions.

Device 502, in the various embodiments and configurations described hereinabove, has been primarily described as a tool for measuring pressure, as represented by the distance moved by a part of the sensor. It is to be noted, however, that an important use for such a device is in the measurement of muscle strength and of muscle tone, for various muscles and muscle groups concentrating around various cavities of the human body. Device 502 can be used to quickly, easily, and consistently measure the strength of such muscle groups. For some diagnostic purposes, the distance of vaginal walls one from another, in various dimensions, can be considered to be correlated with muscular strength of those. For other purposes, measurements may be taken comparing distances in various directions measured without voluntary tensing of muscles, and compared to measurements in the same dimensions taken during tensing of muscles. In some diagnostic contexts, a large difference between the relaxed and the tensed state of the muscles can be taken as a measure of muscular strength. In other diagnostic contexts, a large difference between particular measures of distances between tensed and relaxed states may reflect a weakness in muscles which, had they been stronger, would have prevented such a large displacement.

A fruitful utilization of device 502 consists of measuring change in muscle strength as a response to therapeutic procedures. For one example, urinary incontinence is sometimes treatable by the use of Kegel exercises or other exercises. Yet it is widely known that Kegel exercises are difficult to learn to do properly, and that a large percentage of the patients attempting to treat urinary incontience through the use of such exercises do not reap the expected benefit because they do not properly execute the exercise. In this context, use of device 502 can benefit such patients both by providing immediate feedback during execution of an exercise as to the correctness of the exercise, and by providing long-term feedback to a patient by measuring changes in muscle strength over the course of a treatment program.

As described, urinary incontinence may in many cases be treated by means of electrical stimulation of muscles for the purpose of developing strength of the stimulated muscles and improved muscle tone throughout the urinary tract area. Device 502 may be useful in evaluating progress of such a treatment, by providing consistent measurements of muscle strength, comparable over time. System 500, comprising device 502 and a data utilization system as described hereinabove in the context of a discussion of FIG. 14, is operative to show developmental trends in data recorded over a course of treatment.

Referring again to FIG. 15, there is presented a configuration which is highly convenient when device 502 is used as a measurement device in the context of a course of therapeutic stimulation of muscles to treat urinary incontinence. Electrodes 630 of a therapeutic muscle stimulation system may be combined with the configurations of device 502 as described hereinabove, to provide a combine stimulation and measurement device 632, comprising both measurement configurations of device 502, and muscle stimulation configurations comprising electrodes for electrical stimulation of muscles, and other elements constituting an electrical muscle stimulation device.

Thus, the present embodiments provide inter alia a device utilizable for electrically stimulating the muscles and nerves defining and surrounding a bodily cavity. By virtue of it's memory properties the device, according to the teachings of the invention: (i) self conforms to the anatomy of a wide range of individuals thus maintaining optimal contact between the electrode pairs provided thereupon and the wall of the cavity; (ii) can be easily applied to, and removed from, the cavity; and (iii) it remains at the desired location within the cavity without producing discomfort while allowing the individual to be mobile without fear of device displacement.

The above embodiments thereby enable accurate stimulation of the muscles and nerves defining and surrounding the cavity, and measurement of muscular activity, the measurement of muscular activity allowing biofeedback techniques as described above.

Thus electrical-stimulation and biofeedback monitoring provided by the device of the present invention can be utilized to effectively increase muscle strength while assessing muscle activity and prescribing the best course of treatment for the individual treated.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A displacement measuring device operable to measure distances within a body cavity, comprising:
(a) a flexible container whose shape is subject to deformation by pressure exerted on said container;
(b) an electronic measuring unit contained in said flexible container, operable to respond quantitatively to deformations in the shape of said container; and
(c) an output unit for transmitting data from said measuring device to an external data utilization system.

2. The device of claim 1, further comprising an external data utilization system.

3. The device of claim 1, wherein said container, when not subject to deforming pressure, is of substantially annular shape.

4. The device of claim 1, wherein said container, when not subject to deforming pressure, is of a substantially rounded diamond shape.

5. The device of claim 1, wherein said container comprises a layer of silicon substantially covering and containing said electronic measuring unit.

6. The device of claim 2, wherein said data utilization system comprises a user sensible display.

7. The device of claim 6, wherein said display comprises a bar display.

8. The device of claim 2, wherein said data utilization system comprises a microprocessor.

9. The device of claim 2, wherein said data utilization system comprises a memory.

10. The device of claim 2, wherein said data utilization system comprises a data recording facility.

11. The device of claim 10, wherein said data recording facility comprises a database.

12. The device of claim 2, wherein said data utilization system comprises a biofeedback system.

13. The device of claim 2, wherein said data utilization system comprises a calculation module for comparing data recorded at a first time to data recorded at a second time.

14. The device of claim 13, wherein said data utilization system comprises a trend displayer for displaying to a user trends discerned by said calculation module.

15. The device of claim 1, wherein said output unit comprises a wire connection operable to transmit data between said measuring unit and said external data utilization system.

16. The device of claim 1, wherein said output unit comprises a radio-frequency data transmission module operable to transmit data between said measuring unit and said external data utilization system.

17. The device of claim 1, wherein said output unit comprises a fiber-optic connection operable to transmit data between said measuring unit and said external data utilization system.

18. The device of claim 1, wherein said output unit comprises an infra-red data transmission module operable to transmit data between said measuring unit and said external data utilization system.

19. The device of claim 1, wherein said electronic measuring unit comprises a capacitance measurement tool operable to respond quantitatively to changes in capacitance of a capacitance module, said change in capacitance being induced by variations in the shape of said container.

20. The device of claim 1, wherein said electronic measuring unit comprises an inductance module comprising a tuned oscillator including a coil, and further comprising a metallic element flexibly connected to said coil, said flexible connection being such that changes in shape of said container change a spatial relationship between said metallic element and said coil, thereby modifying inductance of said coil, thereby modifying frequency and phase of oscillation of said tuned circuit.

21. The device of claim 20, wherein said inductance module further comprises a quantification module operable to respond quantitatively to said modification of frequency.

22. The device of claim 20, wherein said inductance module further comprises a quantification module operable to respond quantitatively to said modification of phase.

23. The device of claim 1, wherein said electronic measuring unit comprises a signal strength measurement sensor comprising:
(a) a signal generator operable to generate a generated signal receivable by a signal receiver; and
(b) at least one signal receiver;
said at least one signal receiver being flexibly connected to said signal generator in such a manner that changes to the shape of said container necessarily change a spatial relationship between said signal generator and said signal receiver, thereby modifying a strength of said signal as received by said signal receiver.

24. The device of claim 23, wherein said signal generator is a generator of electronic signals.

25. The device of claim 24, wherein said signal generator is operable to generate a signal of a type selected from a group including pulse signals, wave signals, modulated pulse signals, modulated wave signals, changing pulse signals, changing wave signals, phase shift signals and changing frequency signals.

26. The device of claim 23, wherein said signal generator is an infrared transmitter and said signal receiver is an infrared receiver.

27. The device of claim 23, wherein said signal generator is a light transmitter and said signal receiver is a light receiver.

28. The device of claim 23, wherein said signal generator is a permanent magnet and said signal receiver is a magnetic sensor.

29. The device of claim 28, wherein said magnetic sensor is a Hall effect semiconductor receiver.

30. The device of claim 23, wherein said signal generator is an electro-static field generator utilizing a plate antenna, and said signal receiver is an electro-static field sensor utilizing a plate antenna.

31. The device of claim 23, wherein said signal generator is an ultrasound sound generator, and said signal receiver is an ultrasound sound sensor.

32. The device of claim 23, wherein said signal generator is a loudspeaker, and said signal receiver is a microphone.

33. The device of claim 23, wherein said signal generator comprises a very low radiation isotope, and said signal receiver is a radiation sensor operable to measure an amount of received radiation.

34. The device of claim 1, wherein said electronic measuring unit comprises a compressible gas-filled structure containing a variable-resistance device whose electrical resistance varies as a function of pressure of gas in said compressible gas-filled structure.

35. The device of claim 1, wherein said electronic measuring unit comprises a variable resistor whose resistance is a function of mechanical exerted on said resistor.

36. A muscle strength measuring device for measuring forces exerted on said device by muscles of a body while said device is inserted in a cavity of said body, the device comprising:
(a) a flexible container sized and shaped to fit into a body cavity, said container being operable to undergo shape deformation in response to pressure exerted on said body by contraction of muscles constituting or adjacent to said body cavity; and
(b) an electronic mechanism at least partially contained in said container, operable to measure said deformation of said container in response to said pressure.

37. A distance measuring device for measuring distances within a body cavity, the device comprising:
(a) a flexible container sized and shaped to fit into a body cavity, said container being operable to undergo shape deformation in response to pressure exerted on said body by walls of said body cavity; and
(b) an electronic mechanism at least partially contained in said container, operable to measure said deformation of said container in response to said pressure.

38. The device of claim 1, further comprising electrodes for electrically stimulating muscles of said body.

39. A cavity measuring device operable to measure changes within a body cavity, comprising:
(a) a flexible container whose shape is subject to deformation by pressure exerted on said container;
(b) an electronic measuring unit contained in association with said flexible container, operable to respond quantitatively to changes within said cavity; and
(c) an output unit for transmitting data from said measuring device to an external data utilization system.

40. The device of claim 39, wherein said change is a pressure change.

41. The device of claim 39, wherein said cavity is an animal body cavity and said changes comprises changes in pressure exerted by musculature surrounding said cavity.

42. The device of claim 39, wherein said electronic measuring unit comprises at least one sensor located outwardly of said device.

43. The device of claim 39, wherein said electronic measuring unit comprises at least one sensor located in a cavity within said device.

44. The device of claim 42, wherein said cavity is generally expulsive of foreign objects introduced therein, said flexible container being adapted to deform within said cavity upon being pressurized by said musculature such as to retain itself within said cavity.

45. A method for biofeedback training of a user, comprising (a) inserting into a body cavity a muscle strength measuring device operable to measure forces exerted on said device by muscles of a body while said device is inserted in said body cavity, the device comprising:
(i) a flexible container sized and shaped to fit into a body cavity, said container being operable to undergo shape deformation in response to pressure exerted on said body by contraction of muscles constituting or adjacent to said body cavity;
(ii) a sensor mechanism at least partially contained in said container, operable to measure said deformation of said container in response to said pressure; and
(iii) a biofeedback mechanism operatively associated with said sensor mechanism to provide user sensible feedback of said deformation; and
(b) using said biofeedback to guide muscle exercising.
